(19) United States
(12) Patent Application Publication
(10) Pub. No.: US 2026/0087356 A1
(43) Pub. Date: Mar. 26, 2026

(54) ORDINAL CONTRASTIVE LEARNING FOR IMPUTING MISSING DATA WITH PROGRESSIVE LABELS

(71) Applicant: POSTECH RESEARCH AND BUSINESS DEVELOPMENT FOUNDATION, Pohang-si (KR)

(72) Inventors: Won Hwa KIM, Pohang-si (KR); Seung Hun BAEK, Pohang-si (KR); Jae Yoon SIM, Pohang-si (KR)

(21) Appl. No.: 19/336,903

(22) Filed: Sep. 23, 2025

(30) Foreign Application Priority Data
Sep. 24, 2024 (KR) 10-2024-0128729
Nov. 6, 2024 (KR) 10-2024-0156117

(51) Int. Cl. G06N 3/09; G06N 3/0455; G16H 30/20

(52) U.S. Cl. CPC G06N 3/09; G06N 3/0455; G16H 30/20

(57) ABSTRACT

A method of training a generative model for missing data imputation comprises: acquiring a first dataset; embedding, using an encoder included in the generative model, each of a plurality of data included in the first dataset into an embedding space; reconstructing, using a decoder included in the generative model, each of the plurality of data included in the first dataset based on a result of the embedding; and training the encoder based on the result of the embedding. A method of missing data imputation comprises: acquiring a missing dataset; acquiring a target modality condition; inputting the missing dataset and the target modality condition into a generative model; and imputing missing data based on an output value of the generative model.

### CROSS-REFERENCE TO RELATED APPLICATION

This application claims priority to and the benefit of (i) Korean Patent Application No. 10-2024-0128729, filed on Sep. 24, 2024, and (ii) Korean Patent Application No. 10-2024-0156117, filed on Nov. 6, 2024, each filed in the Korean Intellectual Property Office, under 35 U.S.C. § 119 (a). The entire disclosure of each of the foregoing applications is incorporated herein by reference in its entirety.

### BACKGROUND

### 1. Field

The disclosure relates to methods of imputing missing data

### 2. Discussion of Related Art

Multimodal data may refer to data that integrates different types or formats of data. In the medical field, multimodal data may include CT scan images, a patient's health records (text data), and ECG signals. With advances in artificial neural network (ANN)-based AI models, tasks utilizing multimodal data have become prevalent, such as diagnosing diseases by inputting medical images into AI-based models. However, not all patients have all modalities available, which hinders the development of models that diagnose diseases using medical images and the like. In addition, even beyond diagnostic modeling, various AI tasks may face missing data for certain modalities, which poses challenges in training AI models or performing target tasks using them

### SUMMARY

To diagnose neurodegenerative diseases such as Alzheimer's disease (AD), multimodal imaging data such as MRI and PET are used. Diseases such as Alzheimer's disease are characterized by progression over time. Accordingly, multimodal imaging provides specific information related to the degree of progression of the disease. However, it is rare that every patient has all modality data. Therefore, there has been a difficulty in developing models that diagnose a disease using medical images and the like.

In addition to the development of models that diagnose a disease, in various tasks using AI models, there are also cases in which data of a specific modality are missing. Accordingly, there has been a difficulty in training AI models or in performing intended tasks using AI models.

The technology described below is intended to disclose a method that can accurately impute missing data by utilizing correlations among various modalities in data in which order exists.

### Technical Solution

The technology described below discloses a training method of a generative model for missing-data imputation and a missing-data imputation method.

In one embodiment, a training method of a generative model for missing-data imputation comprises: a step in which an electronic device acquires a first dataset; a step in which the electronic device embeds, into an embedding space, each of a plurality of data included in the first dataset using an encoder included in the generative model; a step in which the electronic device reconstructs, using a decoder included in the generative model, each of a plurality of data included in the first dataset based on respective results of the embedding; and a step in which the electronic device trains the encoder based on the results of the embedding.

In one embodiment, the first dataset comprises data of a plurality of modality types in which order exists.

In one embodiment, the first dataset comprises missing data in which a portion of data is missing.

In one embodiment, training the encoder comprises training the encoder so that an embedding position in the embedding space is determined according to the order of a plurality of data included in the first dataset.

In one embodiment, a missing-data imputation method comprises: a step in which an electronic device acquires a missing dataset; a step in which the electronic device acquires a target modality condition; a step in which the electronic device inputs the missing dataset and the target modality condition into a generative model; and a step in which the electronic device imputes missing data based on an output value of the generative model.

By using the technology described below, missing data can be effectively imputed. By using the technology described below, data for a specific modality type that are missing in multimodality data can be effectively imputed. By using the technology described below, when analyzing data for progressive neurodegenerative diseases such as Alzheimer's disease, data missing among various image data such as MRI, PET, and CT can be imputed.

By using the technology described below, missing data can be imputed while taking into account order included in data. By using the technology described below, missing data can be complemented so that information among a plurality of modality types for the same subject is consistently maintained.

By using the technology described below, missing data that occur in clinical data can be effectively imputed. By using the technology described below, performance of progression-degree analysis and prediction models for neurodegenerative diseases can be greatly improved. By using the technology described below, accuracy of medical image data analysis can be increased, and important contributions can be made to early diagnosis of diseases such as Alzheimer's disease and to establishment of personalized treatment plans.

### BRIEF DESCRIPTION OF THE DRAWINGS

FIG. 1 is an example in which an electronic device 100 performs a training method for a generative model for missing data imputation and a missing data imputation method.

FIG. 2 illustrates an example (200) of the training method for a generative model for missing data imputation.

FIG. 3 illustrates an example (300) of the missing data imputation method.

FIG. 4 illustrates an encoder training process according to an embodiment.

FIG. 5 illustrates a decoder training process according to an embodiment.

FIG. 6 illustrates differences between supervised contrastive learning as prior art and ordinal contrastive learning according to the training method for a generative model for missing data imputation.

FIG. 7 illustrates an example in which the missing data imputation method is performed using the generative model.

FIG. 8 shows an embedding result of ADNI data obtained using an encoder trained by the disclosed training method.

FIG. 9 shows results of ROI-wise statistical analysis after imputation on ADNI data.

FIG. 10 shows an example evaluation of prediction performance for AD progression using multimodal data after performing the disclosed training method.

FIG. 11 shows a configuration example of an electronic device 400.

### DETAILED DESCRIPTION

Since the technology to be described below can have various changes and can have various embodiments, specific embodiments are illustrated in the drawings and described in detail. However, this is not intended to limit the technology described below to specific embodiments, and it should be understood to include all changes, equivalents, and alternatives falling within the spirit and scope of the technology described below.

The terms “first,” “second,” “A,” “B,” etc., may be used to describe various components, but the components are not limited by the terms, which are only used to distinguish one component from another. For example, without departing from the scope of the following description, a first component may be referred to as a second component, and similarly, a second component may also be referred to as a first component. The term “and/or” includes any and all combinations of one or more of the associated listed items.

As used herein, the singular forms should be understood to include the plural forms unless the context clearly indicates otherwise, and the terms “comprises,” “comprising,” “includes,” and/or “including,” when used herein, specify the presence of stated features, integers, steps, operations, elements, components and/or groups thereof, and do not preclude the presence or addition of one or more other features, integers, steps, operations, elements, components and/or groups thereof.

Before describing the drawings in detail, it should be clarified that the division of constituent parts in this specification is merely a division by main functions of each constituent part. That is, two or more constituent parts to be described below may be combined into one constituent part, or one constituent part may be divided into two or more constituent parts for each subdivided function. In addition, each of the constituent parts described below may additionally perform some or all of the functions of other constituent parts in addition to the main function of the constituent part itself, and it goes without saying that some of the main functions performed by each of the constituent parts may be performed exclusively by other components.

Also, in performing a method or an operation method, processes constituting the method may take place differently from the stated order unless clearly specified in the context. That is, each process may occur in the same order as described, may be performed substantially simultaneously, or may be performed in reverse order.

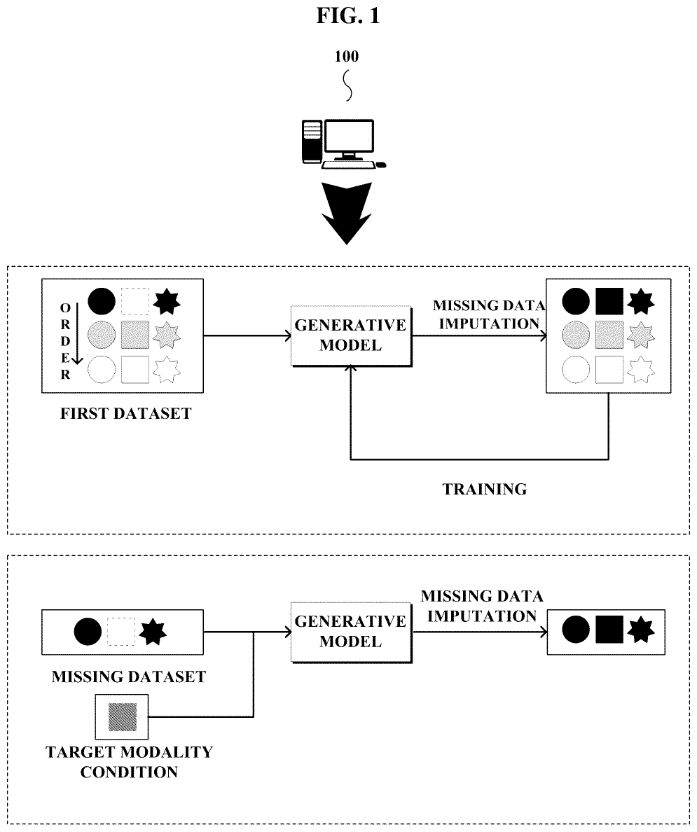

FIG. 1 is one embodiment in which an electronic device (100) performs a training method for a generative model for missing data imputation and a missing data imputation method.

The electronic device (100) may be implemented in various physical forms. For example, the electronic device (100) may take the form of a PC, a notebook, a smart device, a server, or a chipset dedicated to data processing.

At least one electronic device (100) may be present. That is, the training method for a generative model for missing data imputation and the missing data imputation method may be performed by one electronic device, or may be divided and performed by at least one device.

In some embodiments, the electronic device 100 may be configured to perform at least one of: (i) a method of training a generative model for imputing missing data and (ii) a method of imputing missing data using a generative model trained by the foregoing training method. Either or both of the training method and the imputation method may be executed by the electronic device 100. Although a single device may perform both methods, this is not required, and in other embodiments different devices respectively perform the training method and the imputation method (e.g., a server for training and a client device for imputation).

The electronic device (100) may acquire a first dataset. The electronic device (100) may input the first dataset to a generative model. The electronic device (100) may impute missing data in the first dataset based on an output value of the generative model. The electronic device (100) may train the generative model based on data in which missing data have been imputed.

The electronic device (100) may acquire a missing dataset. The electronic device (100) may acquire a target modality condition. The electronic device (100) may input the missing dataset and the target modality condition to a generative model. The electronic device (100) may impute missing data based on an output value of the generative model.

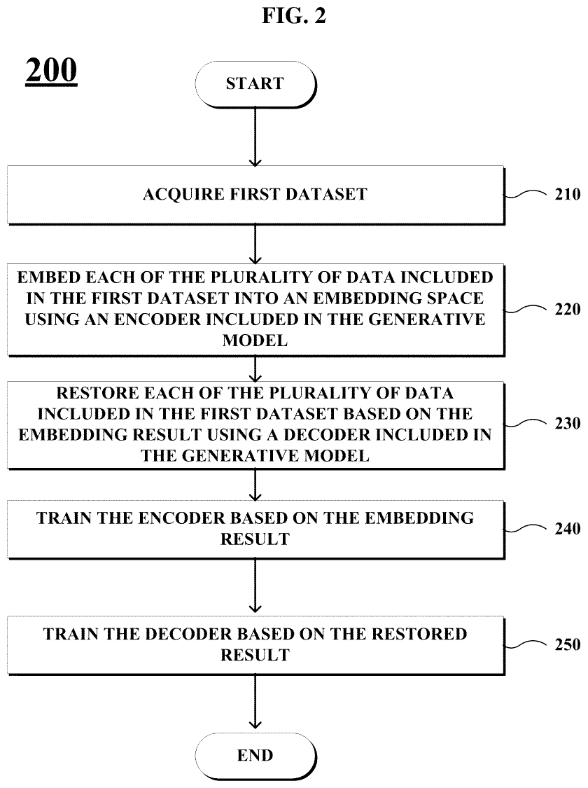

FIG. 2 shows one embodiment (200) of the training method for a generative model for missing data imputation.

In an embodiment, the electronic device may acquire a first dataset (210).

The first dataset may include a plurality of data.

The first dataset may include various kinds of data. For example, the first dataset may be a dataset for medical images.

In some embodiments, the first dataset may include ordered data whose order is determined by a predetermined rule, such as, but not limited to, chronological order (time-ordered data), acquisition sequence, degree of disease progression, or disease severity. The order may be a total or a partial order.

That data have order may mean that distances or similarities among the data can be expressed as order. For example, data of first order and data of second order may be close to or similar to each other; conversely, data of first order and data of seventh order may be far from or dissimilar to each other.

The first dataset may include data of a plurality of modality types. For example, the first dataset may include MRI data, X-ray data, and CT data. Or the first dataset may include image-type data, sound-type data, and text-type data.

The first dataset may include data acquired from a plurality of sources. The first dataset may include data acquired from different sources. The first dataset may include data acquired from different subjects. For example, the first dataset may include 30 medical image data acquired from patient A and 10 medical image data acquired from patient B.

The first dataset may include ordered data of different modality types acquired from different sources. For example, the first dataset may include ten MRI images of early-stage cancer acquired from patient A, five CT images of early-stage cancer acquired from patient A, three PET images of mid-stage cancer acquired from patient A, five MRI images of early-stage cancer acquired from patient B, and two CT images of late-stage cancer acquired from patient B.

The first dataset may include missing data in which a portion of data is missing. For example, if the first dataset includes MRI, CT, and X-ray data of patient A every three days for 30 days, the first dataset may include data in which the MRI data of patient A on the sixth day are missing.

In an embodiment, the electronic device may embed (map), using an encoder included in the generative model, each of a plurality of data included in the first dataset into an embedding space (220).

In an embodiment, the electronic device may reconstruct, using a decoder included in the generative model, each of a plurality of data included in the first dataset based on a result of embedding (230).

The generative model may be a machine learning (ML)-based model. The generative model may be an artificial neural network (ANN)-based model. The ANN may be a deep neural network (DNN), and may include a convolutional neural network (CNN), a recurrent neural network (RNN), a restricted Boltzmann machine (RBM), a deep belief network (DBN), a generative adversarial network (GAN), and relation networks (RL).

The generative model may include an encoder.

The encoder may embed each of a plurality of data included in the first dataset into an embedding space. The encoder may extract features from each of a plurality of data included in the first dataset.

The generative model may include a decoder.

The decoder may reconstruct data based on a result embedded into an embedding space. The decoder may reconstruct each of a plurality of data included in the first dataset based on a result of embedding performed by the encoder for each of a plurality of data included in the first dataset.

The decoder may receive, together with a result embedded into an embedding space, a source modality condition to reconstruct each of a plurality of data included in the first dataset.

The source modality condition may be information indicating in what modality type to reconstruct when reconstructing data. This is because, as will be described later, the encoder does not consider what the modality type of the input data is when extracting features.

According to what source modality condition is input to the decoder, the modality type of the reconstructed data may be determined. In a training process, the source modality condition may be the same as the initial modality type before being input to the generative model. In an inference process, the source modality condition may be information on a modality that is missing. The source modality condition in the inference process may be referred to as a target modality condition.

In an embodiment, the electronic device may train the encoder based on a result of embedding (240).

Training the encoder may include adjusting parameters of the encoder. Training the encoder may include training the encoder using a loss function.

Training the encoder may include enabling the encoder to reflect order of data. Training the encoder may include training the encoder so that data are embedded in an embedding space according to the order of data. Training the encoder may include training the encoder so that positions of data in the embedding space are determined according to the order of data. Training the encoder may include training the encoder so that embedding positions in the embedding space are determined according to the order of a plurality of data included in the first dataset.

In some embodiments, by training the encoder to be order-aware, samples with small differences in order are mapped to nearby points in the embedding space, whereas samples with large differences in order are mapped farther apart.

For example, if data included in the first dataset are data measured at 30-minute intervals for 12 hours, training the encoder may include training the encoder so that the 30-minute order is reflected as a distance between data to be embedded. In this case, data measured at similar times may be located close to each other in the embedding space, and data measured at different times may be located far from each other in the embedding space.

For example, if data included in the first dataset include data according to the degree of disease progression (early, mid, and late cancer), training the encoder may include training the encoder so that the embedding position in the embedding space is determined according to the order of the degree of disease progression. In this case, data for early cancer may be located close to data for mid cancer, but relatively far from data for late cancer.

Training the encoder may include training the encoder so that data for the same source are embedded in similar positions in the embedding space.

For example, if data included in the first dataset are data for patient A and patient B, the encoder may be trained so that data for patient A are located close to each other in the embedding space, but data for patient A and data for patient B are located far from each other.

Training the encoder may include training the encoder not to consider modality types of a plurality of data included in the first dataset.

For example, when data included in the first dataset are MRI data, X-ray data, and CT data, what each modality type is may be made not to affect embedding. Accordingly, the encoder may be trained to embed data according to the degree of disease progression regardless of the modality type. Or, regardless of the modality type, the encoder may be trained so that data acquired from the same subject are embedded in similar positions.

In an embodiment, the electronic device may train the decoder based on the reconstructed result (250).

Training the decoder may include adjusting parameters of the decoder. Training the decoder may include training the decoder using a loss function.

Training the decoder may include training the decoder so that the decoder can well reconstruct the first input data. Accordingly, the decoder may be trained using reconstruction error.

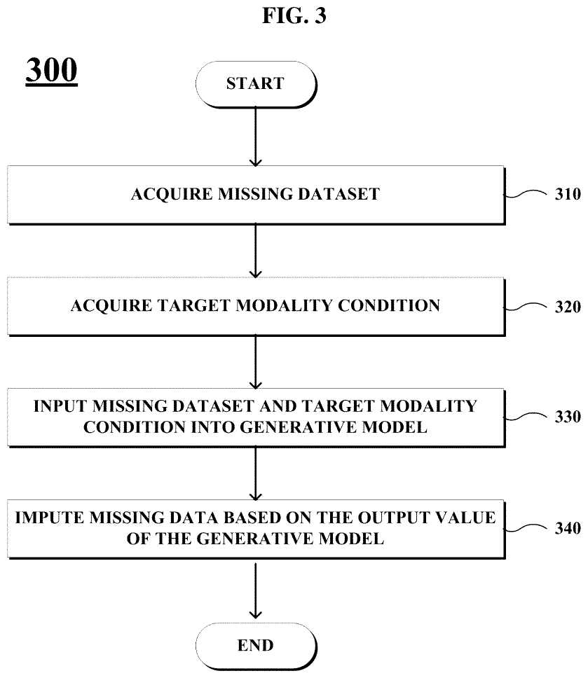

FIG. 3 shows one embodiment (300) of the missing data imputation method.

In an embodiment, the electronic device may acquire a missing dataset (310).

The missing dataset may include at least one data.

The missing dataset may include various kinds of data. For example, the missing dataset may include data for medical images.

The missing dataset may include data of a plurality of modality types. For example, the missing dataset may include MRI data, X-ray data, and CT data. Or the missing dataset may include image-type data, sound-type data, and text-type data.

The missing dataset may include missing data in which a portion of data is missing. For example, if the missing dataset includes MRI, CT, and X-ray data of patient A every three days for 30 days, the missing dataset may include data in which the MRI data of patient A on the sixth day are missing.

In an embodiment, the electronic device may acquire a target modality condition (320).

The target modality condition may include information on the modality of the missing data that are missing in the missing dataset. The target modality condition may include information on in which modality type to impute the missing data missing in the missing dataset. For example, if the missing data are MRI data, the target modality condition may include information that the modality type of the missing data is MRI data.

In an embodiment, the electronic device may input the missing dataset and the target modality condition to the generative model (330).

The generative model may be a model trained through the training method for a generative model for missing data imputation described above. The generative model may impute missing data in the missing dataset. The generative model may include an encoder and a decoder.

The generative model may receive at least one of the data included in the missing dataset as input. The generative model may use at least one data that are received as input to impute missing data.

The generative model may impute missing data so as to satisfy the input target modality condition.

In an embodiment, the electronic device may impute missing data based on an output value of the generative model (340).

Below, an embodiment implementing the training method for a generative model for missing data imputation is described.

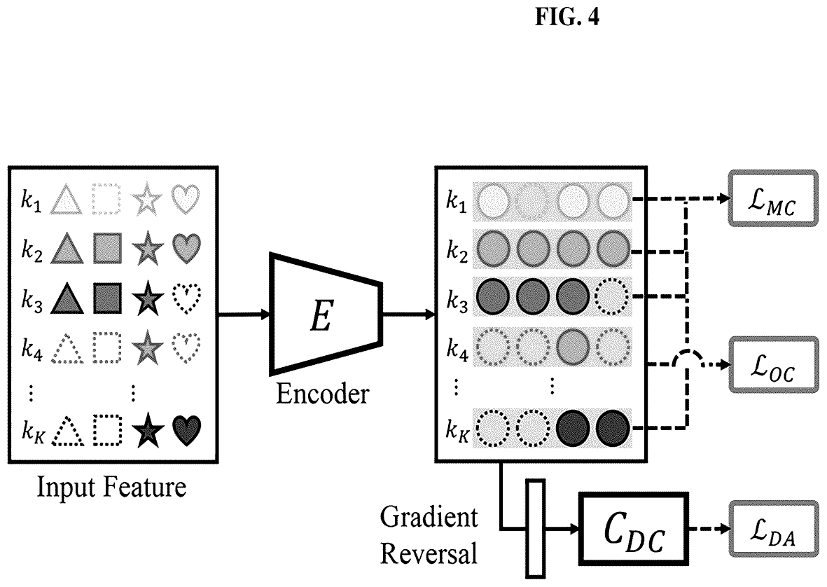

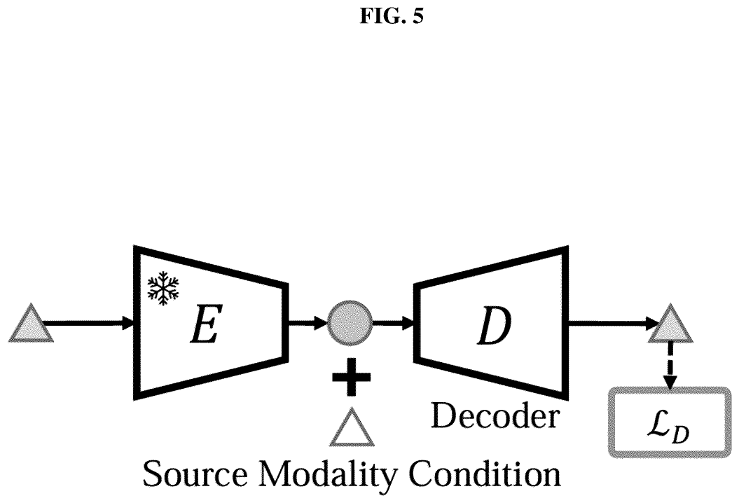

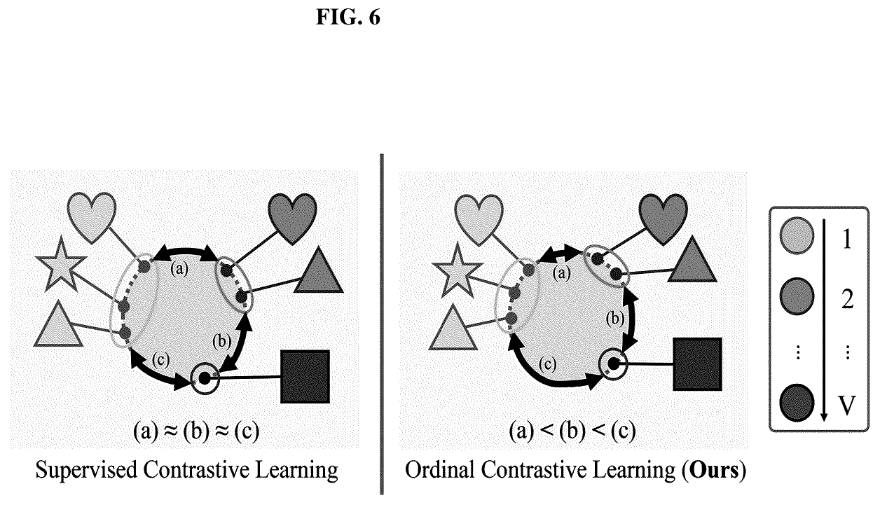

FIG. 4 shows an encoder training process according to an embodiment. FIG. 5 shows a decoder training process according to an embodiment. FIG. 6 is one embodiment showing differences between supervised contrastive learning as prior art and ordinal contrastive learning according to the training method for a generative model for missing data imputation.

Data X include data for K subjects. The data X may be the first dataset described above.

Data included in X have Q-dimensional features. Data for the k-th subject include data for S modalities. Therefore, among the data included in data X, the data for the k-th subject may be expressed as xₖ∈ [symbol] ^{{S×Q}}. And among the data for the k-th subject, the data for the s-th modality may be expressed as x_{{k,s}}∈ [symbol] ^{{Q}};

The data (xₖ) for the k-th subject have label values according to the degree of disease progression or the severity of the disease. Label values according to the degree of disease progression or severity may be expressed as yₖ∈{1, . . . , V}.

An encoder (E) may extract information on the degree of disease progression according to a label value (yₖ) from each modality data (x_{{k,s}}) for all K subjects. The encoder may generate modality-independent embeddings that reflect disease progression. The encoder may also generate embeddings that maintain consistency among multiple modalities within the same patient.

A decoder (D) reconstructs data for each modality based on the embedding result generated by the encoder. The decoder reconstructs data by matching the embedding result to a specific modality. The decoder may receive, through a source modality condition, information on the modality type that has disappeared in the encoder's feature extraction process. The decoder maintains cross-modality consistency and disease progression information.

The encoder may be trained by the following loss functions.

### —Domain Adversarial Training (L_{DA}).

Domain adversarial training is a method that trains the encoder in a direction to remove modality-specific information (modality-specific information associated with s) from the input data. That is, domain adversarial training may include training the encoder not to consider modality types of a plurality of data included in the first dataset.

Domain adversarial training may be performed through a modality classifier CDC and a gradient reversal layer. The gradient reversal layer is located between the encoder and the modality classifier (CDC) and reverses a gradient.

Domain adversarial training may be defined as in Equation (1).

$${ℒ}_{DA} = 𝒥 ( s , {C}_{DC} ( E ( {x}_{k , s} ) ) ) \qquad [ Equation ( 1 ) ]$$

In Equation (1), J may mean an appropriate loss function such as cross-entropy. Through the gradient reversal layer, the encoder may be trained so that L_{DA} is maximized. Through this, the encoder can be trained to remove modality-specific information in the embedding process. At the same time, the modality classifier (CDC) may be trained in a direction in which L_{DA} is minimized.

### —Ordinal Contrastive Learning (L_{OC}).

For the downstream task, the encoder should be able to more accurately extract the degree of disease progression. The encoder should be trained so that each data are embedded in the embedding space reflecting the order according to the degree of disease progression. That is, ordinal contrastive learning may include training the encoder so that data are embedded in the embedding space according to the order of data.

Ordinal contrastive learning may be derived from supervised contrastive learning (SCL). Equation (2) is one of the equations generally used when performing supervised contrastive learning.

$${ℒ}_{SC} = {\sum }_{i ∈ I} \frac{- 1}{\vert P ( i ) \vert} {\sum }_{p ∈ P ( i )} log \frac{exp ( {z}_{i} \cdot {z}_{p} / τ )}{{\sum }_{p ∈ P ( i )} exp ( {z}_{i} \cdot {z}_{p} / τ ) + {\sum }_{n ∈ N ( i )} exp ( {z}_{i} \cdot {z}_{n} / τ )} \qquad [ Equation ( 2 ) ]$$

In Equation (2), i denotes an index of a sample in a batch I. In Equation (2), zᵢ is a result of embedding a sample xᵢ. In Equation (2), zᵢ has a label yᵢ. In Equation (2), P(i) may mean a set of indices of samples having the same label as the i-th sample. P(i)=≡{p∈P(i):ŷₚ=ŷᵢ}. In Equation (2), N(i) may mean a set of indices of samples having a label different from that of the i-th sample. N(i)≡{n∈N(i):ŷₙ·ŷᵢ} In Equation (2), |P(i)| may mean cardinality of a set. In Equation (2), τ may mean a scalar temperature parameter.

In Equation (2), a single τ controlling the strength of separation can operate similarly to classification loss by ignoring the degree of differences between labels. Assuming that label values according to the degree of disease progression are arranged according to the degree of disease progression, a function d(yᵢ, yₙ) measuring the distance between two labels may be defined as |yᵢ−yₙ|. In this case, a larger d(yᵢ, yₙ) means a greater degree of disease progression, which indicates that the difference between zᵢ and zₙ is larger. Therefore, by setting τ(i, n) to τ(i, n)/d(i, n) according to yᵢ and yₙ so that a larger penalty is applied as the distance between labels increases, and by setting an adaptive τ(i, n) for each zₙ and a unique τ(i, P) for each zₚ, an ordinal contrastive loss function (L_{OC}) may be defined as in Equation (3).

$${ℒ}_{OC} = {\sum }_{i ∈ I} \frac{- 1}{\vert P ( i ) \vert} {\sum }_{p ∈ P ( i )} log \frac{exp ( {z}_{i} \cdot {z}_{p} / {τ}_{i , {P}^{′}} )}{{\sum }_{q ∈ P ( i )} exp ( {z}_{i} \cdot {z}_{q} / {τ}_{i , P} ) + {\sum }_{n ∈ N ( i )} exp ( {z}_{i} \cdot {z}_{n} / {τ}_{i , n} )} . \qquad [ Equation ( 3 ) ]$$

To prevent collisions in the embedding space, the gradient magnitudes for positives and negatives should be the same. Therefore, a value τ(i, P) between zᵢ and zₚ may be set as in Equation (4).

$${τ}_{i , P} = \frac{{\sum }_{n ∈ N ( i )} exp ( {z}_{i} , {z}_{{n}_{{i}^{′}}} / {τ}_{i , n} )}{{\sum }_{n ∈ N ( i )} exp ( {z}_{i} , {z}_{{n}_{{i}^{′}}} / {τ}_{i , n} ) / {τ}_{i , n}} . \qquad [ Equation ( 4 ) ]$$

Comparing supervised contrastive learning and ordinal contrastive learning is as shown in FIG. 6.

In the supervised contrastive learning used conventionally, data belonging to the same class are positives, and the remaining data are negatives. Therefore, it does not distinguish according to labels. The conventional method repels all samples with the same intensity without considering order such as disease progression. Therefore, distances for each class do not differ greatly ((a)≈(b)≈(c)), whereas ordinal contrastive learning assigns penalizing strength according to distances between labels. Therefore, distances between data differ according to labels.

—Maximize Modality-Wise Coherence within a Subject (L_{MC}).

The domain adversarial training (L_{DA}) and the ordinal contrastive learning (L_{OC}) described above require only unpaired data. However, data originating from the same subject need to be embedded similarly to each other. In order to maintain consistency of data of different modality types within the same subject, maximizing modality-wise coherence within a subject (L_{MC}) may be used. That is, maximizing modality-wise coherence within a subject may include training the encoder so that data for the same source are embedded in similar positions in the embedding space.

L_{MC} may be implemented through a function such as cosine similarity. Equation (5) is one example of L_{MC}.

$${ℒ}_{MC} = \frac{{\sum }_{k = 1}^{K} {\sum }_{i , j ∈ \{ 1 , ⋯ , S \}} - {δ}_{k} ( i , j ) \cdot sim ( {x}_{k , i} , {x}_{k , j} )}{{\sum }_{k = 1}^{K} {\sum }_{i , {j ∈ \{ 1 , ⋯ , S \}}_{i \neq j}} {δ}_{k} ( i , j )} \qquad [ Equation ( 5 ) ]$$

In Equation (5), sim means a cosine similarity function. In Equation (5), δ_{k(i, j)} refers to an indicator function. δ_{k(i, j)} has a value of 1 if x_{(k, i)} and x_{(k, j)} originated from the same subject k, and 0 if they originated from different subjects.

Summarizing the loss function (L_{E}) for the encoder function is as in Equation (6).

$${ℒ}_{E} = {ℒ}_{DA} + {ℒ}_{OC} + {ℒ}_{MC} \qquad [ Equation ( 6 )$$

The decoder may be trained by the following loss function.

—Modality-Conditioned Reconstruction from Embeddings.

Similar to conditional generation methods, by setting a target modality type t as a one-hot condition vector (cₜ∈ [symbol] ₛ), the decoder estimates a target modality under a given embedding condition. Although there are not many data having all modalities from the same subject, thanks to L_{DA} and L_{MC}, a loss L_{D} of the decoder may be approximated to a self-reconstruction loss of x_{{k,t}} as in Equation (7).

$${ℒ}_{D} ( {x}_{k , s} , {x}_{k , t} ) = {‖ {x}_{k , t} - D ( [ E ( {x}_{k , s} ) , {c}_{i} ] ) ‖}^{2} \approx {ℒ}_{D} ( {x}_{k , t} ) = {‖ {x}_{k , t} - D ( [ E ( {x}_{k , t} ) , {c}_{t} ] ) ‖}^{2} \qquad [ Equation ( 7 ) ]$$

Below, one embodiment of the missing data imputation method is described.

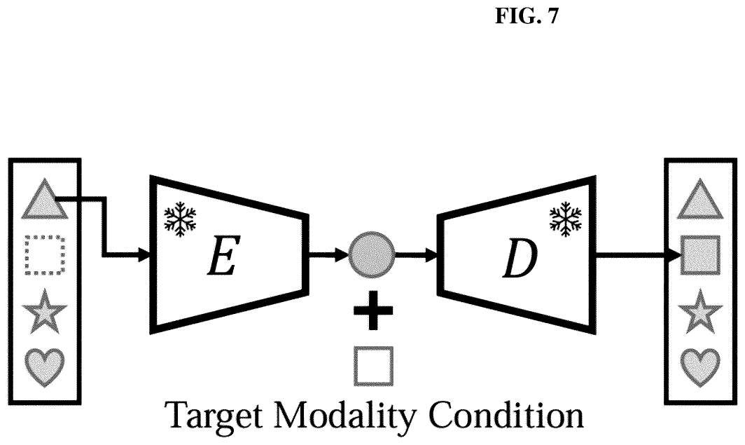

FIG. 7 is one embodiment in which the missing data imputation method is performed using a generative model.

The generative model may receive one of the data included in the missing dataset as input. The generative model may receive a target modality condition as input. The generative model may impute missing data based on the input data and the target modality condition.

Below, results of performance evaluation by implementing the training method for a generative model for missing data imputation and the missing data imputation method are described.

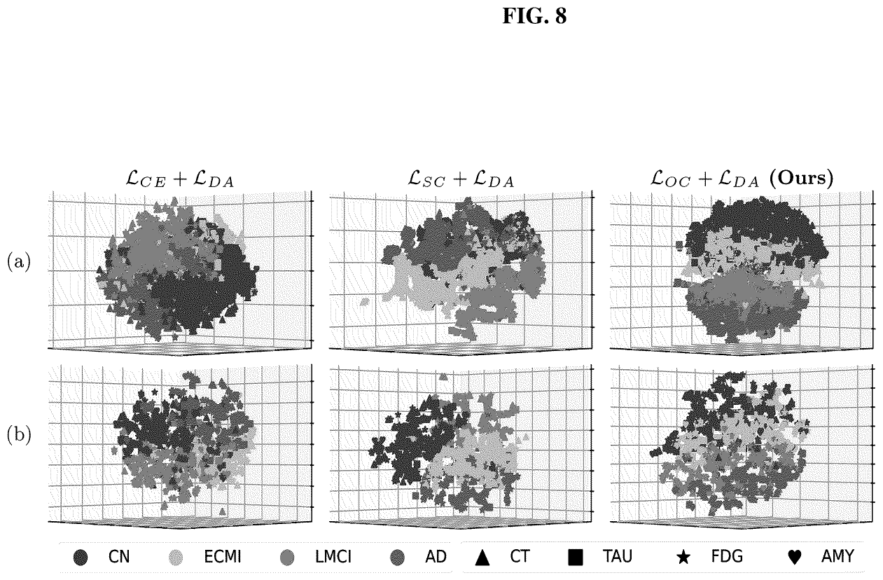

FIG. 8 shows a result of embedding data included in the ADNI (Alzheimer's Disease Neuroimaging Initiative) dataset using an encoder trained by the training method for a generative model for missing data imputation according to one embodiment. FIG. 8 is one result in which an embedding space is visualized using a t-SNE technique.

It can be confirmed that an encoder trained by the training method for a generative model for missing data imputation (Ours) arranges embedded samples according to disease stages more effectively than conventional Cross-Entropy (L_{CE}) and Supervised Contrastive Learning (L_{SC}).

In particular, it can be confirmed that sequential changes according to disease progression are clearly revealed in the embedding space. This shows that it is advantageous for more precisely discriminating each stage of a neurodegenerative disease. This analysis result also shows that the above technique clearly reveals differences between data by embedding reflecting differences in severity.

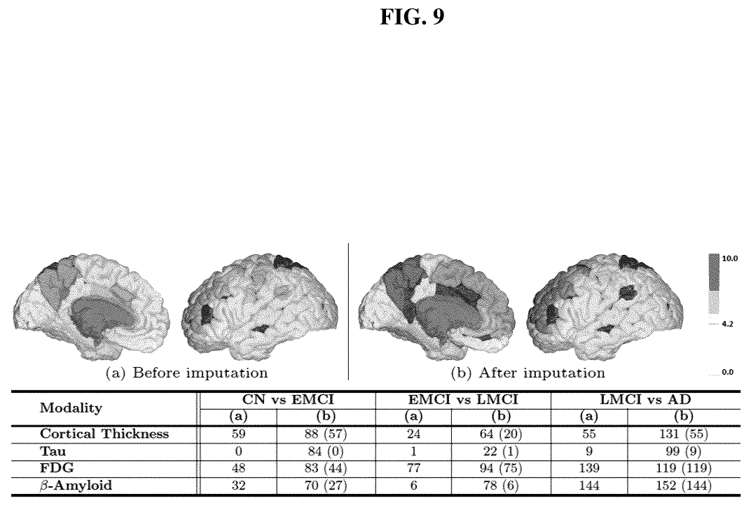

FIG. 9 shows a result of performing statistical analysis by ROI (Regions of Interest) for each brain region in the ADNI dataset after imputing missing data at ADNI through the training method for a generative model for missing data imputation described above.

FIG. 9 shows results of performing group comparison analysis among each disease progression stage (Control (CN), Early Mild Cognitive Impairment (EMCI), Late Mild Cognitive Impairment (LMCI), and Alzheimer's Disease (AD)) using imputed data and real data.

As a result of applying Bonferroni correction (p<0.01), it can be confirmed that imputed data can detect statistically significant changes in a greater number of brain regions than existing data. In particular, for major indices such as Cortical Thickness, Tau, FDG, and Amyloid, imputed data increase statistical sensitivity and can identify significant differences in more ROIs. This confirms that using the method described above effectively imputes missing data, contributing to providing important additional information for analyzing AD progression.

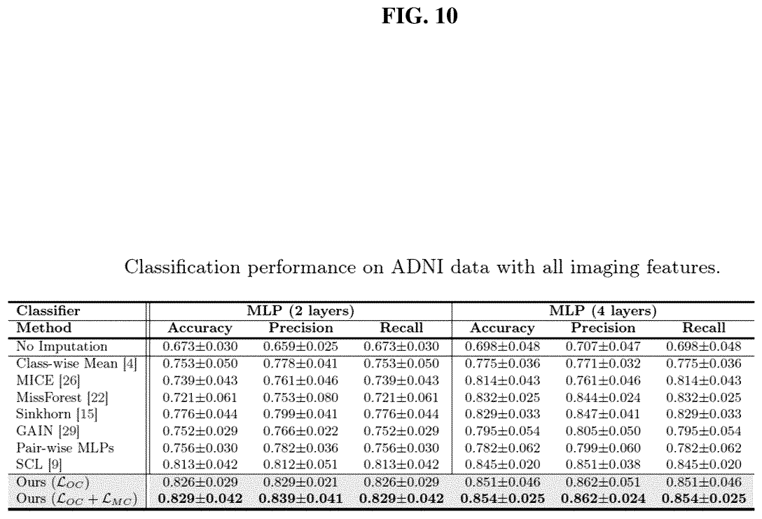

FIG. 10 is one embodiment evaluating prediction performance of AD progression using multimodal data after performing the training method for a generative model for missing data imputation described above.

To predict AD progression stages, a multi-layer perceptron (MLP) model was used. In the ADNI dataset, classification performance was measured using an MLP model for each sample.

In MLP models with 2 layers and 4 layers, it can be confirmed that higher classification accuracy, precision, and recall are obtained when applying the training method for a generative model for missing data imputation than when trained without imputation.

In particular, when using the training method for a generative model for missing data imputation (OCL) described above, accuracy was about 1.3% higher than the existing Supervised Contrastive Learning (SCL) technique, confirming that imputed values reflecting disease progression are effective in improving prediction model performance. In addition, when further applying the LMC (Maximize modality-wise coherence) technique of the training method for a generative model for missing data imputation described above, accuracy of 83% or more was recorded in classification of four disease stages (CN, EMCI, LMCI, AD). This indicates that the training method for a generative model for missing data imputation described above plays an important role in greatly improving prediction model performance.

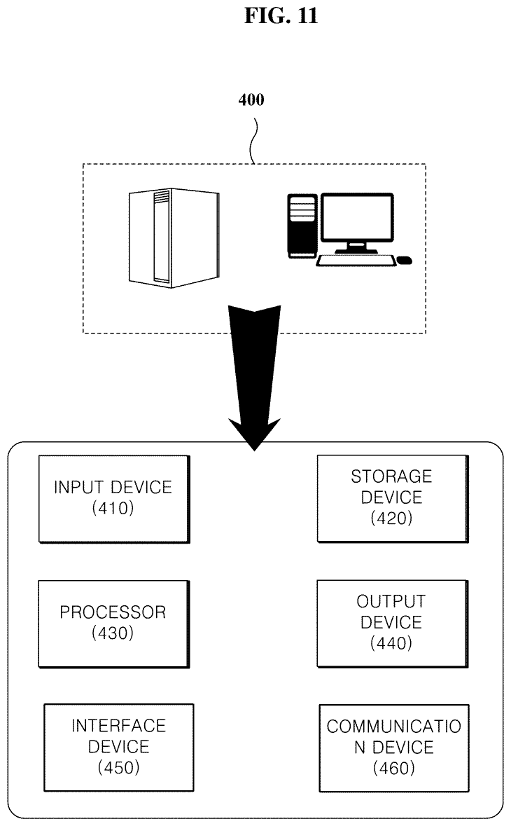

FIG. 11 shows a configuration of one embodiment of an electronic device (400).

The electronic device (400) may correspond to the electronic device (100) described with FIG. 1 and the like. That is, the electronic device (400) may be a device that performs the training method for a generative model for missing data imputation and the missing data imputation method described above.

The electronic device (400) may include at least one input device (410), storage device (420), processor (430), output device (440), interface device (450), and communication device (460).

The input device (410) may receive data, information, or models necessary to perform the training method for a generative model for missing data imputation described above. The input device (410) may receive a first dataset, a missing dataset, a source modality condition and a target modality condition. The input device (410) may receive a generative model. The input device (410) may receive training data necessary to train the generative model. The input device (410) may include a device (keyboard, mouse, touchscreen, joystick, trackball, touchpad, scanner, webcam, etc.) that inputs certain commands or data. The input device (410) may include a configuration that receives data through a separate storage device (USB, CD, hard disk, etc.). The input device (410) may receive data through a separate measuring device or a separate database. The input device (410) may receive data by wire or wirelessly through the communication device (460). The input device (410) may receive a control signal for controlling the electronic device (400).

The storage device (420) may store data, information, or models necessary to perform the training method for a generative model for missing data imputation described above. The storage device (420) may store a first dataset, a missing dataset, a source modality condition and a target modality condition. The storage device (420) may store a generative model. The storage device (420) may store training data necessary to train the generative model. The storage device (420) may be a device that stores certain data, information, or models. The storage device (420) may store data, information, and models received through the input device (410). The storage device (420) may store instructions that cause the processor (430) to perform operations required for the training method for a generative model for missing data imputation. The storage device (420) may store information generated in a process in which the processor (430) performs operations. That is, the storage device (420) may include a memory. For example, the storage device may include an HDD (Hard Disk Drive), an SSD (Solid State Drive), a ROM, a RAM, and CD-ROM, magnetic tape, or a floppy disk.

The processor (430) may perform operations necessary to perform the training method for a generative model for missing data imputation described above.

The processor (430) may acquire a first dataset. The processor (430) may embed, using an encoder included in the generative model, each of a plurality of data included in the first dataset into an embedding space. The processor (430) may reconstruct, using a decoder included in the generative model, each of a plurality of data included in the first dataset based on a result of embedding. The processor (430) may train the encoder based on a result of embedding.

The processor (430) may perform operations necessary to perform the missing data imputation method described above. The processor (430) may acquire a missing dataset. The processor (430) may acquire a target modality condition. The processor (430) may input the missing dataset and the target modality condition to the generative model. The processor (430) may impute missing data based on an output value of the generative model.

The processor (430) may be a device that processes data and processes certain operations, such as a processor, an application processor (AP), and a chip in which a program is embedded. For example, the processor (430) may include a CPU (Central Processing Unit), a GPU (Graphics Processing Unit), or an NPU (Neural Processing Unit). The processor (430) may generate a control signal that controls the electronic device (400). The processor (430) may generate control signals that control the input device (410), the storage device (420), the output device (440), the interface device (450), and the communication device (460) included in the electronic device (400).

The output device (440) may be a device that outputs certain data, information, and models. The output device (440) may be a device that outputs certain data, information, and models to the outside of the electronic device (400). The output device (440) may output interfaces necessary for data processing, input data, analysis results, and the like. The output device (440) may include devices that output data, etc., by tactile, visual, auditory, gustatory, and olfactory methods. The output device (440) may be implemented in various physical forms such as a display, a speaker, a vibration motor, or a document output device. The output device (440) may output data, information, or models stored in the storage device (420). The output device (440) may output data, information, and models generated in a process in which the processor (430) performs operations. The output device (440) may output results calculated by the processor (430).

The interface device (450) may be a device that receives certain commands and data from the outside. The interface device (450) may receive a control signal for controlling the electronic device (400). The interface device (450) may output results analyzed by the electronic device (400). The interface device (450) may receive information necessary to perform the training method for a generative model for missing data imputation described above from a physically connected input device or an external storage device.

The communication device (460) may receive information necessary to perform the training method for a generative model for missing data imputation described above. The communication device (460) may receive a model necessary to perform the training method for a generative model for missing data imputation described above. The communication device (460) may transmit and receive a first dataset, a missing dataset, a source modality condition and a target modality condition. The communication device (460) may transmit and receive a generative model. The communication device (460) may receive a control signal necessary to control the electronic device (400). The communication device (460) may transmit results analyzed by the electronic device (400). The communication device (460) may mean a configuration that receives and transmits certain data, information, and models via a wired or wireless network. The communication device (460) may perform network communication such as Wi-Fi (Wireless Fidelity), Wi-Fi Direct, Bluetooth, UWB (Ultra-Wide Band) or NFC (Near Field Communication), USB (Universal Serial Bus), or HDMI (High Definition Multimedia Interface), and LAN (Local Area Network).

The training method for a generative model for missing data imputation described above may be implemented as a program (or application) including an executable algorithm that can be executed on a computer.

The program may be provided stored on a computer-readable medium that can be read temporarily or non-temporarily.

The temporary readable medium refers to various RAMs such as Static RAM (SRAM), Dynamic RAM (DRAM), Synchronous DRAM (SDRAM), Double Data Rate SDRAM (DDR SDRAM), Enhanced SDRAM (ESDRAM), Synclink DRAM (SLDRAM), and Direct Rambus RAM (DRRAM).

The non-temporary readable medium means a medium that stores data semi-permanently, rather than a medium that stores data for a short moment such as a register, a cache, or a memory, and that can be read by a device. Specifically, the above various applications or programs may be provided stored on a non-temporary readable medium such as a CD, a DVD, a hard disk, a Blu-ray disk, a USB, a memory card, a ROM (read-only memory), a PROM (programmable read-only memory), an EPROM (Erasable PROM, EPROM) or an EEPROM (Electrically EPROM), or a flash memory.

The embodiment herein and the drawings attached to this specification merely clearly show a part of the technical idea included in the above-described technology, and it will be obvious that modifications and specific embodiments that can be easily inferred by those skilled in the art within the scope of the technical idea included in the specification and drawings of the above-described technology are all included in the scope of rights of the above-described technology.

What is claimed is:

1. A method of training a generative model for missing data imputation, the method comprising:
acquiring, by an electronic device, a first dataset;
embedding, by the electronic device using an encoder included in the generative model, each of a plurality of data included in the first dataset into an embedding space;
reconstructing, by the electronic device using a decoder included in the generative model, each of the plurality of data included in the first dataset based on a result of the embedding; and
training, by the electronic device, the encoder based on the result of the embedding,
wherein the first dataset comprises ordered data across a plurality of modality types,
wherein the first dataset is a missing dataset in which a portion of data is missing, and
wherein training the encoder comprises training the encoder such that the positions of the plurality of data in the embedding space are determined based on their order in the first dataset.

2. The method of claim 1, wherein the decoder reconstructs each of the plurality of data included in the first dataset by receiving a source modality condition together with the result of the embedding.

3. The method of claim 1, wherein the first dataset comprises data acquired from a plurality of sources, and wherein training the encoder comprises training the encoder such that data from an identical source among the data included in the first dataset are embedded in similar positions in the embedding space.

4. The method of claim 1, wherein training the encoder comprises training the encoder such that the encoder does not consider modality types of the plurality of data included in the first dataset.

5. The method of claim 1, further comprising training, by the electronic device, the decoder based on the reconstructed result, wherein training the decoder comprises training the decoder such that the decoder reconstructs the original input data.

6. An electronic device comprising:
an input device configured to acquire a first dataset;
a processor configured to:
embed, using an encoder included in a generative model, each of a plurality of data included in the first dataset into an embedding space;
reconstruct, using a decoder included in the generative model, each of the plurality of data included in the first dataset based on a result of the embedding; and
train the encoder based on the result of the embedding;
a storage device configured to store the generative model,
wherein the first dataset comprises ordered data across a plurality of modality types,
wherein the first dataset is a missing dataset in which a portion of data is missing,
wherein training the encoder comprises training the encoder such that the positions of the plurality of data in the embedding space are determined based on their order in the first dataset.

7. The electronic device of claim 6, wherein the decoder reconstructs each of the plurality of data included in the first dataset by receiving a source modality condition together with the result of the embedding.

8. The electronic device of claim 6, wherein the first dataset comprises data acquired from a plurality of sources, and wherein training the encoder comprises training the encoder such that data from an identical source among the data included in the first dataset are embedded in similar positions in the embedding space.

9. The electronic device of claim 6, wherein training the encoder comprises training the encoder such that the encoder does not consider modality types of the plurality of data included in the first dataset.

10. The electronic device of claim 6, wherein the processor is further configured to train the decoder based on the reconstructed result, wherein training the decoder comprises training the decoder such that the decoder reconstructs the original input data.

11. A method of missing data imputation, the method comprising:
acquiring, by an electronic device, a missing dataset;
acquiring, by the electronic device, a target modality condition;
inputting, by the electronic device, the missing dataset and the target modality condition into a generative model; and
imputing, by the electronic device, missing data based on an output value of the generative model,
wherein the generative model is trained by the method of claim 1.

12. The method of claim 11, wherein the missing dataset comprises medical imaging data.
